(19) United States
(12) Patent Application Publication
(10) Pub. No.: US 2008/0299479 A1
(43) Pub. Date: Dec. 4, 2008

(54) Toner compositions

(75) Inventors: Yuhua Tong, Webster, NY (US); Chieh-Min Cheng, Rochester, NY (US)

Correspondence Address: Xerox Corporation (CDFS), 445 Broad Hollow Rd.-Suite 420, Melville, NY, 11747, US

(73) Assignee: Xerox Corporation

(21) Appl. No.: 11/809,124

(22) Filed: May 31, 2007

(51) Int. Cl. G03G 9/087; C07C 67/28

(52) U.S. Cl. 430/111.4; 430/113; 560/204

(57) ABSTRACT

Stabilizer compositions are provided having carboxylic acid groups. By esterifying some of the available carboxylic acid groups, the carboxylic acid number of the stabilizer may be adjusted. Such stabilizers may be utilized, in embodiments, for forming toner compositions.

### CROSS-REFERENCE TO RELATED APPLICATIONS

This application is related to co-pending application entitled “Toner Compositions” filed concurrently herewith, under Express Mail Certificate No. EV929664638US (Attorney Docket No. 20061570-US-NP (1515-81)), the disclosure of which is hereby incorporated by reference in its entirety.

### BACKGROUND

The present disclosure relates to toner processes useful in providing toner suitable for electrostatographic apparatuses, including xerographic apparatuses such as digital, image-on-image, and similar apparatuses.

Numerous processes are known for the preparation of toners, such as, for example, conventional processes wherein a resin is melt kneaded or extruded with a pigment, micronized and pulverized to provide toner particles. There are illustrated in U.S. Pat. Nos. 5,364,729 and 5,403,693, the disclosures of each of which are hereby incorporated by reference in their entirety, methods of preparing toner particles by blending together latexes with pigment particles. Also relevant are U.S. Pat. Nos. 4,996,127, 4,797,339, 4,983,488, and 5,922,501, the disclosures of each of which are hereby incorporated by reference in their entirety.

Toner can also be produced by emulsion aggregation methods. Methods of preparing an emulsion aggregation (EA) type toner are known and toners may be formed by aggregating a colorant with a latex polymer formed by emulsion polymerization. For example, U.S. Pat. No. 5,853,943, the disclosure of which is hereby incorporated by reference in its entirety, is directed to a semi-continuous emulsion polymerization process for preparing a latex by first forming a seed polymer. Other examples of emulsion/aggregation/coalescing processes for the preparation of toners are illustrated in U.S. Pat. Nos. 5,403,693, 5,418,108, 5,364,729, and 5,346,797, the disclosures of each of which are hereby incorporated by reference in their entirety. Other processes are disclosed in U.S. Pat. Nos. 5,527,658, 5,585,215, 5,650,255, 5,650,256 and 5,501,935, the disclosures of each of which are hereby incorporated by reference in their entirety.

Toner systems normally fall into two classes: two component systems, in which the developer material includes magnetic carrier granules having toner particles adhering triboelectrically thereto; and single component systems (SDC), which typically use only toner particles. Placing charge on the particles, to enable movement and development of images via electric fields, is most often accomplished with triboelectricity. Triboelectric charging may occur either by mixing the toner with larger carrier beads in a two component development system or by rubbing the toner between a blade and donor roll in a single component system.

The surface chemistry of toner particles may play a role in a toner's triboelectric charging, cohesion, flow capabilities, imaging adhesion, and other similar properties. Development systems which use triboelectricity to charge toner, whether they be two component (toner and carrier) or single component (toner only), may exhibit nonuniform distribution of charges on the surfaces of the toner particles. This nonuniform charge distribution may result in high electrostatic adhesion because of localized high surface charge densities on the particles. For example, the electrostatic adhesion forces for tribo-charged toner, which are dominated by charged regions on the particle at or near its points of contact with a surface, do not rapidly decrease with decreasing size. This so-called “charge patch” effect makes smaller, triboelectric charged particles much more difficult to develop and control. Triboelectricity may also be unpredictable because of the sensitivity of the materials utilized in forming toner.

Improved methods for producing toner, which minimize sensitivity to relative humidity, decrease the production cycling time, and permit excellent control of the charging of toner particles, remain desirable.

### SUMMARY

The present disclosure provides processes for producing toners and toners produced thereby. In embodiments, the present disclosure provides a process including providing a stabilizer of the following formula:

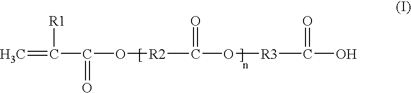

where R1 is a hydrogen or methyl group, R2 and R3 are independently selected from alkyl groups containing about 1 to about 12 carbon atoms and a phenyl group, and n is from about 0 to about 20; contacting the stabilizer with an esterifying agent; and recovering an esterified stabilizer.

In other embodiments, the present disclosure provides a process including providing a stabilizer of the following formula:

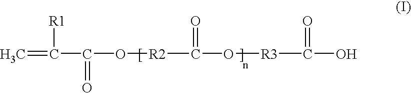

where R1 is a hydrogen or methyl group, R2 and R3 are independently selected from alkyl groups containing about 1 to about 12 carbon atoms and a phenyl group, and n is from about 0 to about 20; contacting the stabilizer with an esterifying agent; recovering an esterified stabilizer; contacting the esterified stabilizer with a latex, a colorant, and an optional wax; and recovering a resulting toner.

In embodiments, the present disclosure also provides a toner including a latex, a colorant, and a stabilizer of the following formula:

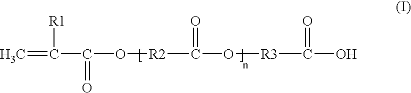

where R1 is a hydrogen or methyl group, R2 and R3 are independently selected from alkyl groups containing about 1 to about 12 carbon atoms and a phenyl group, and n is from about 0 to about 20, wherein carboxylic acid groups of the stabilizer have been esterified so that the stabilizer has a carboxylic acid content from about 0.1 mol/100 g to about 1.4 mol/100 g and the resulting toner particles have a carboxylic acid content from about 0.001 mol/100 g to about 1 mol/100 g.

### DETAILED DESCRIPTION

In accordance with the present disclosure, toner compositions are provided possessing desirable particle sizes. Toner particles of the present disclosure also possess desirable surface charge properties.

Toners of the present disclosure may include a latex resin in combination with a pigment. While the latex resin may be prepared by any method within the purview of one skilled in the art, in embodiments the latex resin may be prepared by current emulsion polymerization methods, including semi-continuous emulsion polymerization, and the toner may include emulsion aggregation toners. Emulsion aggregation involves aggregation of both submicron latex and pigment particles into toner size particles, where the growth in particle size is, for example, in embodiments from about 1 micron to about 15 microns.

### Resin

Any monomer suitable for preparing a latex emulsion can be used in the present processes. Suitable monomers useful in forming the latex emulsion, and thus the resulting latex particles in the latex emulsion include, but are not limited to, styrenes, acrylates, methacrylates, butadienes, isoprenes, acrylic acids, methacrylic acids, acrylonitriles, mixtures thereof, and the like.

In embodiments, the resin of the latex may include at least one polymer. In embodiments, at least one may be from about one to about twenty and, in embodiments, from about three to about ten. Exemplary polymers include copolymers of styrene and acrylates, copolymers of styrene and butadiene, copolymers of styrene and methacrylates, and more specifically, poly(styrene-co-alkyl acrylate), poly(styrene-co-butadiene), poly(styrene-co-alkyl methacrylate), poly(styrene-co-alkyl acrylate-co-acrylic acid), poly(styrene-co-1,3-butadiene-co-acrylic acid), poly(styrene-co-alkyl methacrylate-co-acrylic acid), poly(alkyl methacrylate-co-alkyl acrylate), poly(alkyl methacrylate-co-aryl acrylate), poly(aryl methacrylate-co-alkyl acrylate), poly(alkyl methacrylate-co-acrylic acid), poly(styrene-co-alkyl acrylate-co-acrylonitrile-acrylic acid), poly(styrene-co-butadiene-co-acrylonitrile-co-acrylic acid), poly(alkyl acrylate-co-acrylonitrile-co-acrylic acid), poly(methylstyrene-co-butadiene), poly(methyl methacrylate-co-butadiene), poly(ethyl methacrylate-co-butadiene), poly(propyl methacrylate-co-butadiene), poly(butyl methacrylate-co-butadiene), poly(methyl acrylate-co-butadiene), poly(ethyl acrylate-co-butadiene), poly(propyl acrylate-co-butadiene), poly(butyl acrylate-co-butadiene), poly(styrene-co-isoprene), poly(methylstyrene-co-isoprene), poly(methyl methacrylate-co-isoprene), poly(ethyl methacrylate-co-isoprene), poly(propyl methacrylate-co-isoprene), poly(butyl methacrylate-co-isoprene), poly(methyl acrylate-co-isoprene), poly(ethyl acrylate-co-isoprene), poly(propyl acrylate-co-isoprene), poly(butyl acrylate-co-isoprene), poly(styrene-co-propyl acrylate), poly(styrene-co-butyl acrylate), poly(styrene-co-butadiene-co-methacrylic acid), poly(styrene-co-butyl acrylate-co-acrylic acid), poly(styrene-co-butyl acrylate-co-methacrylic acid), poly(styrene-co-butyl acrylate-co-acrylonitrile), poly(styrene-co-butyl acrylate-co-acrylonitrile-acrylic acid), poly(styrene-co-butyl methacrylate), poly(styrene-co-butyl methacrylate-co-acrylic acid), poly(butyl methacrylate-co-butyl acrylate), poly(butyl methacrylate-co-acrylic acid), poly(acrylonitrile-co-butyl acrylate-co-acrylic acid), and mixtures and combinations thereof. The polymer may be block, random, grafting, or alternating copolymers. In addition, polyester resins obtained from the reaction of bisphenol A and propylene oxide or propylene carbonate, and in particular including such polyesters followed by the reaction of the resulting product with fumaric acid (as disclosed in U.S. Pat. No. 5,227,460, the entire disclosure of which is incorporated herein by reference), and branched polyester resins resulting from the reaction of dimethylterephthalate with 1,3-butanediol, 1,2-propanediol, and pentaerythritol, may also be used.

In embodiments, a poly(styrene-co-butyl acrylate) may be used as the latex resin. The glass transition temperature of this first latex, which in embodiments may be used to form the core of a toner of the present disclosure, may be from about 35° C. to about 75° C., in embodiments from about 40° C. to about 65° C.

In embodiments, the latex may be prepared in an aqueous phase containing a surfactant or co-surfactant. Surfactants which may be utilized in the latex dispersion can be ionic or nonionic surfactants in an amount of from about 0.01 to about 15 weight percent of the solids, and in embodiments of from about 0.1 to about 10 weight percent of the solids.

Anionic surfactants which may be utilized include sulfates and sulfonates, disulfonates, sodium dodecylsulfate (SDS), sodium dodecylbenzene sulfonate, sodium dodecylnaphthalene sulfate, dialkyl benzenealkyl sulfates and sulfonates, acids such as abietic acid available from Aldrich, NEOGEN R™, NEOGEN SC™ obtained from Daiichi Kogyo Seiyaku Co., Ltd., mixtures thereof, and the like. Other suitable surfactants include, in embodiments, DOWFAX™ 2A1, an alkyldiphenyloxide disulfonate from The Dow Chemical Company, optionally in combination with any of the foregoing anionic surfactants.

Examples of cationic surfactants include, but are not limited to, ammoniums, for example, alkylbenzyl dimethyl ammonium chloride, dialkyl benzenealkyl ammonium chloride, lauryl trimethyl ammonium chloride, alkylbenzyl methyl ammonium chloride, alkyl benzyl dimethyl ammonium bromide, benzalkonium chloride, and dodecyl trimethyl ammonium bromides, mixtures thereof, and the like. Other cationic surfactants include cetyl pyridinium bromide, halide salts of quaternized polyoxyethylalkylamines, dodecylbenzyl triethyl ammonium chloride, MIRAPOL and ALKAQUAT available from Alkaril Chemical Company, SANISOL (benzalkonium chloride), available from Kao Chemicals, and the like, and mixtures thereof. In embodiments a suitable cationic surfactant includes SANISOL B-50 available from Kao Corp., which is primarily a benzyl dimethyl alkonium chloride.

Examples of nonionic surfactants include, but are not limited to alcohols, acids and ethers, for example, polyvinyl alcohol, polyacrylic acid, methalose, methyl cellulose, ethyl cellulose, propyl cellulose, hydroxylethyl cellulose, carboxy methyl cellulose, polyoxyethylene cetyl ether, polyoxyethylene lauryl ether, polyoxyethylene octyl ether, polyoxyethylene octylphenyl ether, polyoxyethylene oleyl ether, polyoxyethylene sorbitan monolaurate, polyoxyethylene stearyl ether, polyoxyethylene nonylphenyl ether, dialkylphenoxy poly(ethyleneoxy)ethanol, mixtures thereof, and the like. In embodiments commercially available surfactants from Rhone-Poulenc such as IGEPAL CA-210™, IGEPAL CA-520™, IGEPAL CA-720™, IGEPAL CO-890™, IGEPAL CO-720™, IGEPAL CO-290™, IGEPAL CA-210™, ANTAROX 890™ and ANTAROX 897™ can be selected.

The choice of particular surfactants or combinations thereof as well as the amounts of each to be used are within the purview of those skilled in the art. In embodiments initiators may be added for formation of the latex. Examples of suitable initiators include water soluble initiators, such as ammonium persulfate, sodium persulfate and potassium persulfate, and organic soluble initiators including organic peroxides and azo compounds including Vazo peroxides, such as VAZO 64™, 2-methyl 2-2′-azobis propanenitrile, VAZO 88™, 2-2′-azobis isobutyramide dehydrate, and mixtures thereof. Initiators can be added in suitable amounts, such as from about 0.1 to about 8 weight percent, and in embodiments of from about 0.2 to about 5 weight percent of the monomers.

In embodiments, chain transfer agents may be used including dodecane thiol, octane thiol, carbon tetrabromide, mixtures thereof, and the like, in amounts from about 0.1 to about 10 percent and, in embodiments, from about 0.2 to about 5 percent by weight of monomers, to control the molecular weight properties of the polymer when emulsion polymerization is conducted in accordance with the present disclosure.

### Stabilizers

In embodiments, it may be advantageous to include a stabilizer when forming the toner. Suitable stabilizers include monomers having carboxylic acid functionality. Toner particles produced with such stabilizers may thus possess active functional groups on the particle surface, in embodiments the acid groups. In embodiments, suitable stabilizers may be of the following formula (I):

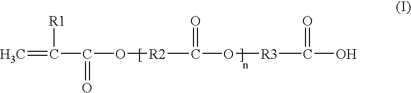

where R1 is hydrogen or a methyl group; R2 and R3 are independently selected from alkyl groups containing from about 1 to about 12 carbon atoms or a phenyl group; and n is from about 0 to about 20, in embodiments from about 1 to about 10. Examples of such stabilizers include beta-carboxyethyl acrylate (sometimes referred to herein as poly(2-carboxyethyl)acrylate) (β-CEA), poly(2-carboxyethyl)methacrylate, poly(3-carboxypropyl)acrylate, poly(4-carboxybutyl)acrylate, 2-carboxyethyl methacrylate, combinations thereof, and the like. Other stabilizers which may be used include, for example, acrylic acid and its derivatives.

In embodiments, the stabilizer having carboxylic acid functionality may also contain metallic ions, such as sodium, potassium and/or calcium, to achieve better emulsion polymerization results. The metallic ions may be present in an amount from about 0.05 to about 10 percent by weight of the stabilizer having carboxylic acid functionality, in embodiments from about 0.1 to about 5 percent by weight of the stabilizer having carboxylic acid functionality.

One potential issue which may arise with the use of the above stabilizers is the variability which may occur in the formation of multiple batches of stabilizers. Since surface properties of toner particles may strongly depend on the stabilizers, the consistency of the quality of the stabilizers may influence toner production. For example, β-CEA may be produced from acrylic acid through a Michael addition reaction. Although reaction temperature can be an important factor in the carboxylic acid number of the β-CEA, with a higher temperature resulting in less carboxylic acid groups, in some cases with the same process time, the Michael reaction can proceed at room temperature, at a much lower reaction rate, resulting in more carboxylic acid groups.

The quality of the β-CEA may thus be inconsistent from batch to batch, especially with respect to the variability in the number of carboxylic acid groups which may result, in part, from different processing temperatures and time. For example, when β-CEA contains more carboxylic acid groups, latexes produced with such stabilizers may possess a larger particle size, which may interfere with the formation of toner particles in an emulsion aggregation process. Poor quality β-CEA may not only cause problems with latex synthesis, including lower quality yield, wider latex particle size distribution, shorter latex shelf life, more reactor fouling, and difficulties in controlling reaction temperature due to higher exothermic reactions, but poor quality and/or variable quality β-CEA may also produce toner particles having unpredictable properties. For example, a β-CEA possessing more carboxylic acid groups may produce toners with higher carboxylic acid content on the surface of the toner particles which, in turn, may influence triboelectric charging, cohesion, flowing capability, imaging adhesion and other physical properties of the toner particles. For example, higher carboxylic acid content on the surface of toner particles may induce unstable triboelectric charging, higher cohesion, lower flowing capability, and increased imaging adhesion which may cause lower transfer efficiency. Thus, it may be desirable to have a uniform and consistent carboxylic acid content in a stabilizer such as β-CEA which, in turn, may assist in providing stable, high quality toners.

In accordance with the present disclosure the carboxylic acid number can be controlled in a stabilizer possessing carboxylic acid groups. While the following discussion focuses on β-CEA, the methods disclosed herein may be used with any stabilizer possessing carboxylic acid groups noted above.

β-CEA includes a series of oligomers. One end of the chain is a carbon-carbon double bond, while the other end of the chain is a carboxylic acid. In embodiments, the carboxylic acid group on a stabilizer such as β-CEA can be esterified to adjust the carboxylic acid content of the stabilizer. For example, the carboxylic acid groups of a stabilizer may be esterified with an alcohol which may, in embodiments, be referred to as an esterifying agent, in which an acid or other suitable catalyst may be used. Suitable alcohols for such an esterification reaction include, but are not limited to, methanol, propanol, butanol, phenylpropyl alcohol, combinations thereof, and the like.

In some embodiments, it may be desirable to use a catalyst when conducting the above esterification reactions. Suitable catalysts are within the purview of those skilled in the art and include, in embodiments, methylsulfuric acid, toluene sulfonic acid, concentrated sulfuric acid, alkylbenzene sulfonic acid, dodecylbenzene sulfonic acid (DBSA), combinations thereof, and the like.

By controlling the amount of alcohol utilized, a defined number of carboxylic acid groups can be converted into ester groups, thereby permitting one to adjust the number of available carboxylic acid groups in β-CEA or a similar stabilizer. Methods for conducting the above esterification reaction are within the purview of those skilled in the art. In embodiments, the amount of alcohol added to the stabilizer may be from about 0.1% to about 99% by weight of the total reaction mixture, in embodiments from about 1% to about 80% by weight of the total reaction mixture, so that the weight ratio of stabilizer to alcohol may be from about 99.9:0.1 to about 1:99, in embodiments from about 99:1 to about 20:80. Where utilized, the amount of catalyst added to the stabilizer in combination with the alcohol may be from about 0.1% to about 20%, in embodiments from about 1% to about 10%. In embodiments, heat may be applied to the esterification reaction so that the reaction takes place at a temperature of from about 25° C. to about 200° C., in embodiments from about 50° C. to about 150° C.

In embodiments, if the carboxylic acid content in a batch of stabilizer is higher than desired, the extra carboxylic acid group can be esterified following the esterification reaction depicted below:

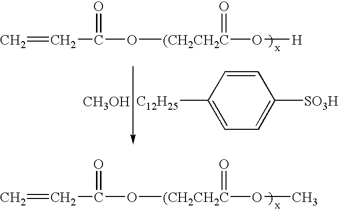

The carboxylic acid content in β-CEA can be measured utilizing methods within the purview of those skilled in the art, including liquid chromatography with UV detection (LC/UV), liquid chromatography/mass spectrometry (LC/MS), liquid chromatography and tandem mass spectrometry (LC/MS/MS), titration, element analysis, nuclear magnetic resonance spectroscopy, combinations thereof, and the like.

In embodiments, the resulting esterified β-CEA may have a carboxylic acid content from about 0.1 mol/100 g to about 1.4 mol/100 g, in embodiments from about 0.2 mol/100 g to about 1 mol/100 g, in other embodiments about 0.72 mol/100 g.

After esterification, the Michael reaction in β-CEA can be stopped. This can solve the storage aging problem for β-CEA. In embodiments, the esterification reaction can be stopped by neutralization with the addition of a base. Moreover, by simple neutralization with a base, including metal hydroxides such as sodium hydroxide, potassium hydroxide, ammonium hydroxide, combinations thereof, and the like, the catalyst, such as a DBSA catalyst, can be converted into sodium dodecylbenzene sulfate which, as noted above, is a surfactant which may be utilized in the latex preparation. Thus, no undesirable side-products are generated in this process which would require a separate removal step.

The resulting β-CEA or similar stabilizer possessing carboxylic acid groups may thus be consistent in the number of available carboxylic acid groups it contains. The consistency in the number of available carboxylic acid groups may result in more uniform and stable toner products produced with the stabilizer, including emulsion aggregation toners. When added to the latex, the stabilizer may provide carboxylic acid functional groups or similar acid groups on the surface of the latex particles which, in turn, may be present on toner particles produced with such latex.

In embodiments, the stabilizer may be present in amounts from about 0.01 to about 15 percent by weight of the resulting toner, in embodiments from about 0.05 to about 5 percent by weight of the toner. Toners possessing these stabilizers may have a carboxylic acid content from about 0.001 mol/100 g to about 1 mol/100 g, in embodiments from about 0.01 mol/100 g to about 0.8 mol/100 g.

### pH Adjustment Agent

In some embodiments a pH adjustment agent may be added to control the rate of the emulsion aggregation process. The pH adjustment agent utilized in the processes of the present disclosure can be any acid or base that does not adversely affect the products being produced. Suitable bases can include metal hydroxides, such as sodium hydroxide, potassium hydroxide, ammonium hydroxide, and optionally mixtures thereof. Suitable acids include nitric acid, sulfuric acid, hydrochloric acid, citric acid, acetic acid, and optionally mixtures thereof.

### Wax

Wax dispersions may also be added to a latex to produce toners of the present disclosure. Suitable waxes include, for example, submicron wax particles in the size range of from about 50 to about 1000 nanometers, in embodiments of from about 100 to about 500 nanometers in volume average diameter, suspended in an aqueous phase of water and an ionic surfactant, nonionic surfactant, or mixtures thereof. Suitable surfactants include those described above. The ionic surfactant or nonionic surfactant may be present in an amount of from about 0.1 to about 20 percent by weight, and in embodiments of from about 0.5 to about 15 percent by weight of the wax.

The wax dispersion according to embodiments of the present disclosure may include, for example, a natural vegetable wax, natural animal wax, mineral wax, and/or synthetic wax. Examples of natural vegetable waxes include, for example, carnauba wax, candelilla wax, Japan wax, and bayberry wax. Examples of natural animal waxes include, for example, beeswax, punic wax, lanolin, lac wax, shellac wax, and spermaceti wax. Mineral waxes include, for example, paraffin wax, microcrystalline wax, montan wax, ozokerite wax, ceresin wax, petrolatum wax, and petroleum wax. Synthetic waxes of the present disclosure include, for example, Fischer-Tropsch wax, acrylate wax, fatty acid amide wax, silicone wax, polytetrafluoroethylene wax, polyethylene wax, polypropylene wax, and mixtures thereof.

Examples of polypropylene and polyethylene waxes include those commercially available from Allied Chemical and Baker Petrolite, including POLYWAX 725®, a polyethylene wax from Baker Petrolite, wax emulsions available from Michelman Inc. and the Daniels Products Company, EPOLENE N-15 commercially available from Eastman Chemical Products, Inc., VISCOL 550-P, a low weight average molecular weight polypropylene available from Sanyo Kasei K.K., and similar materials. In embodiments, commercially available polyethylene waxes possess a molecular weight (Mw) of from about 100 to about 5000, and in embodiments of from about 250 to about 2500, while the commercially available polypropylene waxes have a molecular weight of from about 200 to about 10,000, and in embodiments of from about 400 to about 5000.

In embodiments, the waxes may be functionalized. Examples of groups added to functionalize waxes include amines, amides, imides, esters, quaternary amines, and/or carboxylic acids. In embodiments, the functionalized waxes may be acrylic polymer emulsions, for example, JONCRYL 74, 89, 130, 537, and 538, all available from SC Johnson Wax, or chlorinated polypropylenes and polyethylenes commercially available from Allied Chemical, Petrolite Corporation, and SC Johnson Wax.

The wax may be present in an amount of from about 0.1 to about 30 percent by weight, and in embodiments from about 2 to about 20 percent by weight of the toner.

In the emulsion aggregation process, the reactants may be added to a suitable reactor, such as a mixing vessel. The appropriate amount of at least two monomers, in embodiments from about two to about ten monomers, stabilizer of the present disclosure, surfactant(s), initiator, if any, chain transfer agent, if any, and wax, if any, and the like may be combined in the reactor and the emulsion aggregation process may be allowed to begin. Reaction conditions selected for effecting the emulsion polymerization include temperatures of, for example, from about 45° C. to about 120° C., in embodiments from about 60° C. to about 90° C.

Nanometer size particles may be formed, from about 50 nm to about 800 nm in volume average diameter, in embodiments from about 100 nm to about 400 nm in volume average diameter as determined, for example, by a Brookhaven nanosize particle analyzer.

After formation of the latex particles, the latex particles may be used to form a toner. In embodiments, the toners are an emulsion aggregation type toner that are prepared by the aggregation and fusion of the latex particles of the present disclosure with a colorant, and one or more additives such as stabilizers of the present disclosure, surfactants, coagulants, waxes, surface additives, and optionally mixtures thereof.

### Colorant

The latex particles may be added to a colorant dispersion. The colorant dispersion may include, for example, submicron colorant particles in a size range of, for example, from about 50 to about 500 nanometers and, in embodiments, of from about 100 to about 400 nanometers in volume average diameter. The colorant particles may be suspended in an aqueous water phase containing an anionic surfactant, a nonionic surfactant, or mixtures thereof. In embodiments, the surfactant may be ionic and may be from about 0.1 to about 25 percent by weight, and in embodiments from about 1 to about 15 percent by weight, of the colorant.

Colorants useful in forming toners in accordance with the present disclosure include pigments, dyes, mixtures of pigments and dyes, mixtures of pigments, mixtures of dyes, and the like. The colorant may be, for example, carbon black, cyan, yellow, magenta, red, orange, brown, green, blue, violet, or mixtures thereof.

In embodiments wherein the colorant is a pigment, the pigment may be, for example, carbon black, phthalocyanines, quinacridones or RHODAMINE B™ type, red, green, orange, brown, violet, yellow, fluorescent colorants, and the like.

The colorant may be present in the toner of the disclosure in an amount of from about 1 to about 25 percent by weight of toner, in embodiments in an amount of from about 2 to about 15 percent by weight of the toner.

Exemplary colorants include carbon black like REGAL 330® magnetites; Mobay magnetites including MO8029™, MO8060™; Columbian magnetites; MAPICO BLACKS™ and surface treated magnetites; Pfizer magnetites including CB4799™, CB5300™, CB5600™, MCX6369™; Bayer magnetites including, BAYFERROX 8600™, 8610™; Northern Pigments magnetites including, NP-604™, NP-608™; Magnox magnetites including TMB-100™, or TMB-104™, HELIOGEN BLUE L6900™, D6840™, D7080™, D7020™, PYLAM OIL BLUE™, PYLAM OIL YELLOW™, PIGMENT BLUE 1™ available from Paul Uhlich and Company, Inc.; PIGMENT VIOLET 1™, PIGMENT RED 48™, LEMON CHROME YELLOW DCC 1026™, E.D. TOLUIDINE RED™ and BON RED C™ available from Dominion Color Corporation, Ltd., Toronto, Ontario; NOVAPERM YELLOW FGL™, HOSTAPERM PINK E™ from Hoechst; and CINQUASIA MAGENTA™ available from E.I. DuPont de Nemours and Company. Other colorants include 2,9-dimethyl-substituted quinacridone and anthraquinone dye identified in the Color Index as CI-60710, CI Dispersed Red 15, diazo dye identified in the Color Index as CI 26050, CI Solvent Red 19, copper tetra(octadecyl sulfonamido) phthalocyanine, x-copper phthalocyanine pigment listed in the Color Index as CI 74160, CI Pigment Blue, Anthrathrene Blue identified in the Color Index as CI 69810, Special Blue X-2137, diarylide yellow 3,3-dichlorobenzidene acetoacetanilides, a monoazo pigment identified in the Color Index as CI 12700, CI Solvent Yellow 16, a nitrophenyl amine sulfonamide identified in the Color Index as Foron Yellow SE/GLN, CI Dispersed Yellow 33,2,5-dimethoxy-4-sulfonanilide phenylazo-4′-chloro-2,5-dimethoxy acetoacetanilide, Yellow 180 and Permanent Yellow FGL. Organic soluble dyes having a high purity for the purpose of color gamut which may be utilized include Neopen Yellow 075, Neopen Yellow 159, Neopen Orange 252, Neopen Red 336, Neopen Red 335, Neopen Red 366, Neopen Blue 808, Neopen Black X53, Neopen Black X55, wherein the dyes are selected in various suitable amounts, for example from about 0.5 to about 20 percent by weight, in embodiments, from about 5 to about 20 weight percent of the toner.

In embodiments, colorant examples include Pigment Blue 15:3 having a Color Index Constitution Number of 74160, Magenta Pigment Red 81:3 having a Color Index Constitution Number of 45160:3, Yellow 17 having a Color Index Constitution Number of 21105, and known dyes such as food dyes, yellow, blue, green, red, magenta dyes, and the like.

In other embodiments, a magenta pigment, Pigment Red 122 (2,9-dimethylquinacridone), Pigment Red 185, Pigment Red 192, Pigment Red 202, Pigment Red 206, Pigment Red 235, Pigment Red 269, and the like, and combinations thereof, may be utilized as the colorant.

The resulting blend of latex, optionally in a dispersion, and colorant dispersion may be stirred and heated to a temperature of from about 35° C. to about 70° C., in embodiments of from about 40° C. to about 65° C., resulting in toner aggregates of from about 2 microns to about 10 microns in volume average diameter, and in embodiments of from about 5 microns to about 8 microns in volume average diameter.

### Coagulants

In embodiments, a coagulant may be added during or prior to aggregating the latex and the aqueous colorant dispersion. The coagulant may be added over a period of time from about 1 to about 60 minutes, in embodiments from about 1.25 to about 20 minutes, depending on the processing conditions.

Examples of coagulants include polyaluminum halides such as polyaluminum chloride (PAC), or the corresponding bromide, fluoride, or iodide, polyaluminum silicates such as polyaluminum sulfo silicate (PASS), and water soluble metal salts including aluminum chloride, aluminum nitrite, aluminum sulfate, potassium aluminum sulfate, calcium acetate, calcium chloride, calcium nitrite, calcium oxylate, calcium sulfate, magnesium acetate, magnesium nitrate, magnesium sulfate, zinc acetate, zinc nitrate, zinc sulfate and the like. One suitable coagulant is PAC, which is commercially available and can be prepared by the controlled hydrolysis of aluminum chloride with sodium hydroxide. Generally, PAC can be prepared by the addition of two moles of a base to one mole of aluminum chloride. The species is soluble and stable when dissolved and stored under acidic conditions if the pH is less than about 5. The species in solution is believed to be of the formula Al₁₃O₄(OH)₂₄(H₂O)₁₂ with about 7 positive electrical charges per unit.

In embodiments, suitable coagulants include a polymetal salt such as, for example, polyaluminum chloride (PAC), polyaluminum bromide, or polyaluminum sulfosilicate. The polymetal salt can be in a solution of nitric acid, or other diluted acid solutions such as sulfuric acid, hydrochloric acid, citric acid or acetic acid. The coagulant may be added in amounts from about 0.01 to about 5 percent by weight of the toner, and in embodiments from about 0.1 to about 3 percent by weight of the toner.

### Aggregating Agents

Any aggregating agent capable of causing complexation might be used in forming toner of the present disclosure. Both alkali earth metal or transition metal salts can be utilized as aggregating agents. In embodiments, alkali (II) salts can be selected to aggregate sodio sulfonated polyester colloids with a colorant to enable the formation of a toner composite. Such salts include, for example, beryllium chloride, beryllium bromide, beryllium iodide, beryllium acetate, beryllium sulfate, magnesium chloride, magnesium bromide, magnesium iodide, magnesium acetate, magnesium sulfate, calcium chloride, calcium bromide, calcium iodide, calcium acetate, calcium sulfate, strontium chloride, strontium bromide, strontium iodide, strontium acetate, strontium sulfate, barium chloride, barium bromide, barium iodide, and optionally mixtures thereof. Examples of transition metal salts or anions which may be utilized as aggregating agent include acetates of vanadium, niobium, tantalum, chromium, molybdenum, tungsten, manganese, iron, ruthenium, cobalt, nickel, copper, zinc, cadmium or silver; acetoacetates of vanadium, niobium, tantalum, chromium, molybdenum, tungsten, manganese, iron, ruthenium, cobalt, nickel, copper, zinc, cadmium or silver; sulfates of vanadium, niobium, tantalum, chromium, molybdenum, tungsten, manganese, iron, ruthenium, cobalt, nickel, copper, zinc, cadmium or silver; and aluminum salts such as aluminum acetate, aluminum halides such as polyaluminum chloride, mixtures thereof, and the like.

Neutralizing bases that may be utilized in the toner formulation processes include bases such as metal hydroxides, including sodium hydroxide, potassium hydroxide, ammonium hydroxide, and optionally mixtures thereof. Also useful as a neutralizer is a composition containing sodium silicate dissolved in sodium hydroxide. As noted above, the use of these neutralizing bases can convert a catalyst such as DBSA into sodium dodecylbenzene sulfate, which is a surfactant in the latex preparation. Thus, no undesirable side-products are generated in this process which would require a separate removal step.

### Additives

The toner may also include charge additives in effective amounts of, for example, from about 0.1 to about 10 weight percent, in embodiments from about 0.5 to about 7 weight percent. Suitable charge additives include alkyl pyridinium halides, bisulfates, the charge control additives of U.S. Pat. Nos. 3,944,493; 4,007,293; 4,079,014; 4,394,430 and 4,560,635, the entire disclosures of each of which are hereby incorporated by reference in their entirety, negative charge enhancing additives like aluminum complexes, any other charge additives, mixtures thereof, and the like.

Further optional additives include any additive to enhance the properties of toner compositions. Included are surface additives, color enhancers, etc. Surface additives that can be added to the toner compositions after washing or drying include, for example, metal salts, metal salts of fatty acids, colloidal silicas, metal oxides, strontium titanates, mixtures thereof, and the like, which additives are each usually present in an amount of from about 0.1 to about 10 weight percent, in embodiments from about 0.5 to about 7 weight percent of the toner. Examples of such additives include, for example, those disclosed in U.S. Pat. Nos. 3,590,000, 3,720,617, 3,655,374 and 3,983,045, the disclosures of each of which are hereby incorporated by reference in their entirety. Other additives include zinc stearate and AEROSIL R972® available from Degussa. The coated silicas of U.S. Pat. No. 6,190,815 and U.S. Pat. No. 6,004,714, the disclosures of each of which are hereby incorporated by reference in their entirety, can also be selected in amounts, for example, of from about 0.05 to about 5 percent by weight, in embodiments from about 0.1 to about 2 percent by weight of the toner, which additives can be added during the aggregation or blended into the formed toner product.

Once the appropriate final size of the toner particles is achieved, the pH of the mixture may be adjusted with a base to a value of from about 3.5 to about 7, and in embodiments from about 4 to about 6.5. The base may include any suitable base such as, for example, alkali metal hydroxides such as, for example, sodium hydroxide, potassium hydroxide, and ammonium hydroxide. The alkali metal hydroxide may be added in amounts from about 0.1 to about 30 percent by weight of the mixture, in embodiments from about 0.5 to about 15 percent by weight of the mixture.

The resultant blend of latex, optionally in a dispersion, stabilizer of the present disclosure, optional wax, colorant dispersion, optional coagulant, and optional aggregating agent, may then be stirred and heated to a temperature below the Tg of the latex, in embodiments from about 30° C. to about 70° C., in embodiments of from about 40° C. to about 65° C., for a period of time from about 0.2 hours to about 6 hours, in embodiments from about 0.3 hour to about 5 hours.

In embodiments, a shell may then be formed on the aggregated particles. Any latex utilized noted above to form the core latex may be utilized to form the shell latex. In embodiments, a styrene-n-butyl acrylate copolymer may be utilized to form the shell latex. In embodiments, the latex utilized to form the shell may have a glass transition temperature of from about 35° C. to about 75° C., in embodiments from about 40° C. to about 70° C.

Where used, the shell latex may be applied by any method within the purview of those skilled in the art, including dipping, spraying, and the like. The shell latex may be applied until the desired final size of the toner particles is achieved, in embodiments from about 2 microns to about 10 microns, in other embodiments from about 4 microns to about 8 microns. In other embodiments, the toner particles may be prepared by in-situ seeded semi-continuous emulsion copolymerization of the latex in which the alkaline resin may be added during shell synthesis. Thus, in embodiments, the toner particles may be prepared by in-situ seeded semi-continuous emulsion copolymerization of styrene and n-butyl acrylate (BA), in which calcium resinate may be introduced at the later stage of reaction for the shell synthesis.

The mixture of latex, colorant, optional wax, and any additives, is subsequently coalesced. Coalescing may include stirring and heating at a temperature of from about 80° C. to about 99° C., for a period of from about 0.5 to about 12 hours, and in embodiments from about 1 to about 6 hours. Coalescing may be accelerated by additional stirring.

In embodiments, the pH of the mixture may then be lowered to from about 3.5 to about 6 and, in embodiments, to from about 3.7 to about 5.5 with, for example, an acid, to further coalesce the toner aggregates. Suitable acids include, for example, nitric acid, sulfuric acid, hydrochloric acid, citric acid or acetic acid. The amount of acid added may be from about 0.1 to about 30 percent by weight of the mixture, and in embodiments from about 1 to about 20 percent by weight of the mixture.

The mixture is cooled, washed and dried. Cooling may be at a temperature of from about 20° C. to about 40° C., in embodiments from about 22° C. to about 30° C. over a period time from about 1 hour to about 8 hours, and in embodiments from about 1.5 hours to about 5 hours.

In embodiments, cooling a coalesced toner slurry includes quenching by adding a cooling media such as, for example, ice, dry ice and the like, to effect rapid cooling to a temperature of from about 20° C. to about 40° C., and in embodiments of from about 22° C. to about 30° C. Quenching may be feasible for small quantities of toner, such as, for example, less than about 2 liters, in embodiments from about 0.1 liters to about 1.5 liters. For larger scale processes, such as for example greater than about 10 liters in size, rapid cooling of the toner mixture is not feasible nor practical, neither by the introduction of a cooling medium into the toner mixture, nor by the use of jacketed reactor cooling.

The toner slurry may then be washed. The washing may be carried out at a pH of from about 7 to about 12, and in embodiments at a pH of from about 9 to about 11. The washing may be at a temperature of from about 30° C. to about 70° C., and in embodiments from about 40° C. to about 67° C. The washing may include filtering and reslurrying a filter cake including toner particles in deionized water. The filter cake may be washed one or more times by deionized water, or washed by a single deionized water wash at a pH of about 4 wherein the pH of the slurry is adjusted with an acid, and followed optionally by one or more deionized water washes.

Drying may be carried out at a temperature of from about 35° C. to about 75° C., and in embodiments of from about 45° C. to about 60° C. The drying may be continued until the moisture level of the particles is below a set target of about 1% by weight, in embodiments of less than about 0.7% by weight.

The toner of the present disclosure may have particles with a circularity of from about 0.9 to about 0.99, and in embodiments of from about 0.94 to about 0.98. When the spherical toner particles have a circularity in this range, the spherical toner particles remaining on the surface of the image holding member pass between the contacting portions of the imaging holding member and the contact charger, the amount of deformed toner is small, and therefore generation of toner filming can be prevented so that a stable image quality without defects can be obtained over a long period.

### Uses

Toner in accordance with the present disclosure can be used in a variety of imaging devices including printers, copy machines, and the like. The toners generated in accordance with the present disclosure are excellent for imaging processes, especially xerographic processes, which may operate with a toner transfer efficiency in excess of about 90 percent, such as those with a compact machine design without a cleaner or those that are designed to provide high quality colored images with excellent image resolution, acceptable signal-to-noise ratio, and image uniformity. Further, toners of the present disclosure can be selected for electrophotographic imaging and printing processes such as digital imaging systems and processes.

The imaging process includes the generation of an image in an electronic printing apparatus and thereafter developing the image with a toner composition of the present disclosure. The formation and development of images on the surface of photoconductive materials by electrostatic means is well known. The basic xerographic process involves placing a uniform electrostatic charge on a photoconductive insulating layer, exposing the layer to a light and shadow image to dissipate the charge on the areas of the layer exposed to the light and developing the resulting latent electrostatic image by depositing on the image a finely-divided electroscopic material referred to in the art as “toner”. The toner will normally be attracted to the discharged areas of the layer, thereby forming a toner image corresponding to the latent electrostatic image. This powder image may then be transferred to a support surface such as paper. The transferred image may subsequently be permanently affixed to the support surface as by heat.

Developer compositions can be prepared by mixing the toners obtained with the embodiments of the present disclosure with known carrier particles, including coated carriers, such as steel, ferrites, and the like. See, for example, U.S. Pat. Nos. 4,937,166 and 4,935,326, the disclosures of each of which are hereby incorporated by reference in their entirety. The toner-to-carrier mass ratio of such developers may be from about 2 to about 20 percent, and in embodiments from about 2.5 to about 5 percent of the developer composition. The carrier particles can include a core with a polymer coating thereover, such as polymethylmethacrylate (PMMA), having dispersed therein a conductive component like conductive carbon black. Carrier coatings include silicone resins such as methyl silsesquioxanes, fluoropolymers such as polyvinylidene fluoride, mixtures of resins not in close proximity in the triboelectric series such as polyvinylidene fluoride and acrylics, thermosetting resins such as acrylics, mixtures thereof and other known components.

Development may occur via discharge area development. In discharge area development, the photoreceptor is charged and then the areas to be developed are discharged. The development fields and toner charges are such that toner is repelled by the charged areas on the photoreceptor and attracted to the discharged areas. This development process is used in laser scanners.

Development may be accomplished by the magnetic brush development process disclosed in U.S. Pat. No. 2,874,063, the disclosure of which is hereby incorporated by reference in its entirety. This method entails the carrying of a developer material containing toner of the present disclosure and magnetic carrier particles by a magnet. The magnetic field of the magnet causes alignment of the magnetic carriers in a brush like configuration, and this “magnetic brush” is brought into contact with the electrostatic image bearing surface of the photoreceptor. The toner particles are drawn from the brush to the electrostatic image by electrostatic attraction to the discharged areas of the photoreceptor, and development of the image results. In embodiments, the conductive magnetic brush process is used wherein the developer comprises conductive carrier particles and is capable of conducting an electric current between the biased magnet through the carrier particles to the photoreceptor.

### Imaging

Imaging methods are also envisioned with the toners disclosed herein. Such methods include, for example, some of the above patents mentioned above and U.S. Pat. Nos. 4,265,990, 4,858,884, 4,584,253 and 4,563,408, the entire disclosures of each of which are incorporated herein by reference. The imaging process includes the generation of an image in an electronic printing magnetic image character recognition apparatus and thereafter developing the image with a toner composition of the present disclosure. The formation and development of images on the surface of photoconductive materials by electrostatic means is well known. The basic xerographic process involves placing a uniform electrostatic charge on a photoconductive insulating layer, exposing the layer to a light and shadow image to dissipate the charge on the areas of the layer exposed to the light, and developing the resulting latent electrostatic image by depositing on the image a finely-divided electroscopic material, for example, toner. The toner will normally be attracted to those areas of the layer, which retain a charge, thereby forming a toner image corresponding to the latent electrostatic image. This powder image may then be transferred to a support surface such as paper. The transferred image may subsequently be permanently affixed to the support surface by heat. Instead of latent image formation by uniformly charging the photoconductive layer and then exposing the layer to a light and shadow image, one may form the latent image by directly charging the layer in image configuration. Thereafter, the powder image may be fixed to the photoconductive layer, eliminating the powder image transfer. Other suitable fixing means such as solvent or overcoating treatment may be substituted for the foregoing heat fixing step.

The following Examples are being submitted to illustrate embodiments of the present disclosure. These Examples are intended to be illustrative only and are not intended to limit the scope of the present disclosure. Also, parts and percentages are by weight unless otherwise indicated.

### EXAMPLES

### Example 1

Esterification of β-CEA. In a 1 liter reactor, about 600 grams of β-CEA (from Bimax Chemicals Ltd., carboxylic acid content of about 0.73 mol/100 g) was mixed with about 2 grams of methanol and about 9 grams of dodecylbenzene sulfonic acid. The reactor was heated to a temperature of about 86° C. for about 24 hours. After that time, the reactor was cooled to about 23° C., at which time about 1 gram of NaOH was added to neutralize the catalyst. The resulting esterified β-CEA was ready for addition to a latex in an emulsion aggregation process.

### Example 2

Emulsion aggregation latex synthesis with esterified β-CEA. A monomer mixture was prepared by combining about 206.55 grams of styrene, about 63.45 grams of n-butyl acrylate, about 8.1 grams of esterified β-CEA from Example 1 above, about 0.95 grams of 1,10-decanediol diacrylate, about 4.16 grams of 1-dodecanethiol, about 7.75 grams of DOWFAX™ 2A1 (an alkyldiphenyloxide disulfonate anionic surfactant from The Dow Chemical Company) and about 128.5 grams of deionized water.

About 257 grams of deionized water and about 0.35 grams of DOWFAX™ 2A1 were added to a 1 liter reactor. With mechanical stirring and nitrogen gas flowing, the reactor was heated to about 75° C. About 1% of the above monomer mixture was added. About 10 minutes after the addition, about 4.05 grams of an ammonium persulfate initiator in about 20 grams of water was added over a period of about 10 minutes. About 20 minutes after the addition of this initiator solution, the rest of the monomer mixture was added over about 3 hours. After the addition of the remaining monomer mixture, the resulting milky solution was stirred for about another 3 hours at about 75° C. Then, the latex was cooled to about 23° C. The particle size of the resulting latex, which was determined by a Honeywell MICROTRAC® UPA light scattering instrument, was about 205 nm.

Using the initial β-CEA described above in Example 1 as a control, carboxylic acid content of about 0.73 mol/100 g, which had not been subjected to the esterification reaction of Example 1) the synthesis described above for this Example 2 was utilized to produce a second latex. The particle size of the latex using the original, non-esterified β-CEA was about 280 nm, which was too large for emulsion aggregation toner processes.

### Example 3

Toner Particles Made by Latex with Esterified B-CEA. About 338 grams of the latex produced in Example 2, i.e., the latex with esterified β-CEA, was added to about 63.25 grams of a PR-122 pigment dispersion, about 15.81 grams of a PR-185 pigment dispersion, about 51.2 grams of a POLYWAX 725® polyethylene wax dispersion (M_{W} of about 725, about 31 percent active, commercially available from Baker-Petrolite Company), and about 1056 grams of deionized water. The materials were stirred at about 6400 rpm for about 5 minutes. About 3.46 grams of polyaluminum chloride (DelPAC 2000 from Delta Chemical Corporation) in about 31 grams of about 0.02 N HNO₃ was added slowly. The resulting viscous mixture was then stirred at about 6400 rpm for about 3 more minutes. The mixture was then heated to about 51° C. and stirred at about 500 rpm for about 30 minutes. About 82.71 grams of the latex with esterified β-CEA from Example 2 was then slowly added into the mixture. The resulting slurry was then heated to a temperature of about 51° C. and stirred at a rate of about 450 rpm for about 25 minutes, and then the pH of the slurry was adjusted by the addition of about 4% NaOH solution to obtain a pH of about 5.1. Following the pH adjustment, the slurry was heated to about 96° C. The pH of the solution was then adjusted by the addition of about 0.3M HNO₃ solution to a pH of about 4.2. The particle slurry was stirred at this temperature for about 3 hours at about 400 rpm. The circularity of the resulting particles was obtained by a Sysmex SPIA-2100 (commercially available from Sysmex Co., Kobe, Japan), and found to be about 0.971. After cooling to a temperature from about 20° C. to about 26° C., this slurry was filtered and washed with deionized water. The properties of this toner particle are listed in Table 1 below.

TABLE 1
Toner Particle Properties

| | Median Particle | | | |
|---|---|---|---|---|
| Toner | size by | Median Particle size by | Number | Volume |
| particle | volume, micron | number, micron | GSD | GSD |
| Example 3 | 6.3 | 5.43 | 1.283 | 1.213 |

GSD: geometric size distribution

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A process comprising:
providing a stabilizer of the following formula: 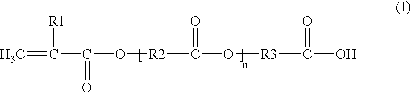
where R1 is a hydrogen or methyl group; R2 and R3 are independently selected from alkyl groups containing about 1 to about 12 carbon atoms and a phenyl group; and n is from about 0 to about 20;
contacting the stabilizer with an esterifying agent; and
recovering an esterified stabilizer.

2. The process of claim 1, wherein the stabilizer is selected from the group consisting of beta carboxyethyl acrylate, poly(2-carboxyethyl)methacrylate, poly(3-carboxypropyl)acrylate, poly(4-carboxybutyl)acrylate, 2-carboxyethyl methacrylate, and combinations thereof, and the esterifying agent is selected from the group consisting of alcohols, acids, and combinations thereof.

3. The process of claim 1, wherein the esterifying agent is selected from the group consisting of methanol, propanol, butanol, phenylpropyl alcohol, and combinations thereof.

4. The process of claim 1, wherein contacting the stabilizer with an esterifying agent further comprises contacting the stabilizer and esterifying agent with a catalyst selected from the group consisting of methylsulfuric acid, toluene sulfonic acid, concentrated sulfuric acid, alkylbenzene sulfonic acid, dodecylbenzene sulfonic acid, and combinations thereof.

5. The process of claim 1, wherein the weight ratio of stabilizer to esterifying agent is from about 99.9:0.1 to about 1:99.

6. The process of claim 1, wherein contacting the stabilizer and esterifying agent takes place at a temperature of from about 25° C. to about 200° C.

7. A process comprising:
providing a stabilizer of the following formula: 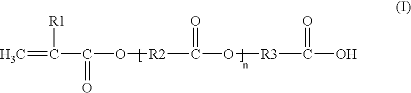
where R1 is a hydrogen or methyl group; R2 and R3 are independently selected from alkyl groups containing about 1 to about 12 carbon atoms and a phenyl group; and n is from about 0 to about 20;
contacting the stabilizer with an esterifying agent;
recovering an esterified stabilizer;
contacting the esterified stabilizer with a latex, a colorant, and an optional wax; and
recovering a resulting toner.

8. The process of claim 7, wherein the stabilizer is selected from the group consisting of beta carboxyethyl acrylate, poly(2-carboxyethyl)methacrylate, poly(3-carboxypropyl)acrylate, poly(4-carboxybutyl)acrylate, 2-carboxyethyl methacrylate, and combinations thereof, and the esterifying agent is selected from the group consisting of methanol, propanol, butanol, phenylpropyl alcohol, and combinations thereof.

9. The process of claim 7, further comprising contacting the stabilizer and esterifying agent with a catalyst selected from the group consisting of methylsulfuric acid, toluene sulfonic acid, concentrated sulfuric acid, alkylbenzene sulfonic acid, dodecylbenzene sulfonic acid, and combinations thereof.

10. The process of claim 7, wherein the weight ratio of stabilizer to esterifying agent is from about 99.9:0.1 to about 1:99, and contacting the stabilizer and esterifying agent takes place at a temperature of from about 25° C. to about 200° C.

11. The process of claim 7, wherein the weight ratio of stabilizer to esterifying agent is from about 99:1 to about 20:80, and contacting the stabilizer and esterifying agent takes place at a temperature of from about 50° C. to about 150° C.

12. The process of claim 7, wherein the latex is selected from the group consisting of styrenes, acrylates, methacrylates, butadienes, isoprenes, acrylic acids, methacrylic acids, acrylonitriles, and combinations thereof.

13. The process of claim 7, wherein the latex is selected from the group consisting of poly(styrene-co-alkyl acrylate), poly(styrene-co-butadiene), poly(styrene-co-alkyl methacrylate), poly(styrene-co-alkyl acrylate-co-acrylic acid), poly(styrene-co-1,3-butadiene-co-acrylic acid), poly(styrene-co-alkyl methacrylate-co-acrylic acid), poly(alkyl methacrylate-co-alkyl acrylate), poly(alkyl methacrylate-co-aryl acrylate), poly(aryl methacrylate-co-alkyl acrylate), poly(alkyl methacrylate-co-acrylic acid), poly(styrene-co-alkyl acrylate-co-acrylonitrile-acrylic acid), poly(styrene-co-butadiene-co-acrylonitrile-co-acrylic acid), poly(alkyl acrylate-co-acrylonitrile-co-acrylic acid), poly(methylstyrene-co-butadiene), poly(methyl methacrylate-co-butadiene), poly(ethyl methacrylate-co-butadiene), poly(propyl methacrylate-co-butadiene), poly(butyl methacrylate-co-butadiene), poly(methyl acrylate-co-butadiene), poly(ethyl acrylate-co-butadiene), poly(propyl acrylate-co-butadiene), poly(butyl acrylate-co-butadiene), poly(styrene-co-isoprene), poly(methylstyrene-co-isoprene), poly(methyl methacrylate-co-isoprene), poly(ethyl methacrylate-co-isoprene), poly(propyl methacrylate-co-isoprene), poly(butyl methacrylate-co-isoprene), poly(methyl acrylate-co-isoprene), poly(ethyl acrylate-co-isoprene), poly(propyl acrylate-co-isoprene), poly(butyl acrylate-co-isoprene), poly(styrene-co-propyl acrylate), poly(styrene-co-butyl acrylate), poly(styrene-co-butadiene-co-methacrylic acid), poly(styrene-co-butyl acrylate-co-acrylic acid), poly(styrene-co-butyl acrylate-co-methacrylic acid), poly(styrene-co-butyl acrylate-co-acrylonitrile), poly(styrene-co-butyl acrylate-co-acrylonitrile-acrylic acid), poly(styrene-co-butyl methacrylate), poly(styrene-co-butyl methacrylate-co-acrylic acid), poly(butyl methacrylate-co-butyl acrylate), poly(butyl methacrylate-co-acrylic acid), poly(acrylonitrile-co-butyl acrylate-co-acrylic acid), and combinations thereof.

14. The process as in claim 7, wherein the toner comprises particles having a volume average diameter of from about 2 microns to about 10 microns, and a circularity from about 0.9 to about 0.99.

15. The process as in claim 7, wherein the toner particles have a carboxylic acid content from about 0.1 mol/100 g to about 1.4 mol/100 g.

16. A toner comprising a latex, a colorant, and a stabilizer of the following formula: 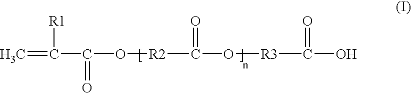
where R1 is a hydrogen or methyl group; R2 and R3 are independently selected from alkyl groups containing about 1 to about 12 carbon atoms and a phenyl group; and n is from about 0 to about 20,
wherein carboxylic acid groups of the stabilizer have been esterified so that the stabilizer has a carboxylic acid content from about 0.1 mol/100 g to about 1.4 mol/100 g and the resulting toner particles have a carboxylic acid content from about 0.001 mol/100 g to about 1 mol/100 g.

17. The toner of claim 16, wherein the latex is selected from the group consisting of styrenes, acrylates, methacrylates, butadienes, isoprenes, acrylic acids, methacrylic acids, acrylonitriles, and combinations thereof, and the stabilizer is selected from the group consisting of beta carboxyethyl acrylate, poly(2-carboxyethyl)methacrylate, poly(3-carboxypropyl)acrylate, poly(4-carboxybutyl)acrylate, 2-carboxyethyl methacrylate, and combinations thereof.

18. The toner of claim 16, wherein the latex is selected from the group consisting of poly(styrene-co-alkyl acrylate), poly(styrene-co-butadiene), poly(styrene-co-alkyl methacrylate), poly(styrene-co-alkyl acrylate-co-acrylic acid), poly(styrene-co-1,3-butadiene-co-acrylic acid), poly(styrene-co-alkyl methacrylate-co-acrylic acid), poly(alkyl methacrylate-co-alkyl acrylate), poly(alkyl methacrylate-co-aryl acrylate), poly(aryl methacrylate-co-alkyl acrylate), poly(alkyl methacrylate-co-acrylic acid), poly(styrene-co-alkyl acrylate-co-acrylonitrile-acrylic acid), poly(styrene-co-butadiene-co-acrylonitrile-co-acrylic acid), poly(alkyl acrylate-co-acrylonitrile-co-acrylic acid), poly(methylstyrene-co-butadiene), poly(methyl methacrylate-co-butadiene), poly(ethyl methacrylate-co-butadiene), poly(propyl methacrylate-co-butadiene), poly(butyl methacrylate-co-butadiene), poly(methyl acrylate-co-butadiene), poly(ethyl acrylate-co-butadiene), poly(propyl acrylate-co-butadiene), poly(butyl acrylate-co-butadiene), poly(styrene-co-isoprene), poly(methylstyrene-co-isoprene), poly(methyl methacrylate-co-isoprene), poly(ethyl methacrylate-co-isoprene), poly(propyl methacrylate-co-isoprene), poly(butyl methacrylate-co-isoprene), poly(methyl acrylate-co-isoprene), poly(ethyl acrylate-co-isoprene), poly(propyl acrylate-co-isoprene), poly(butyl acrylate-co-isoprene), poly(styrene-co-propyl acrylate), poly(styrene-co-butyl acrylate), poly(styrene-co-butadiene-co-methacrylic acid), poly(styrene-co-butyl acrylate-co-acrylic acid), poly(styrene-co-butyl acrylate-co-methacrylic acid), poly(styrene-co-butyl acrylate-co-acrylonitrile), poly(styrene-co-butyl acrylate-co-acrylonitrile-acrylic acid), poly(styrene-co-butyl methacrylate), poly(styrene-co-butyl methacrylate-co-acrylic acid), poly(butyl methacrylate-co-butyl acrylate), poly(butyl methacrylate-co-acrylic acid), poly(acrylonitrile-co-butyl acrylate-co-acrylic acid), and combinations thereof.

19. The toner of claim 16, wherein the toner comprises particles having a volume average diameter of from about 2 microns to about 10 microns, and a circularity from about 0.9 to about 0.99.

20. The process as in claim 16, wherein the toner particles have a carboxylic acid content from about 0.01 mol/100 g to about 0.8 mol/100 g.
